(19) United States
(12) Patent Application Publication
(10) Pub. No.: US 2010/0072062 A1
(43) Pub. Date: Mar. 25, 2010

(54) Membrane For Use With Amperometric Sensors

(75) Inventor: Kenneth M. Curry, Oceanside, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/436,013

(22) Filed: May 5, 2009

Related U.S. Application Data
(60) Provisional application No. 61/050,550, filed on May 5, 2008.

(51) Int. Cl. C12Q 1/54; G01N 27/26

(52) U.S. Cl. 204/403.11; 204/415; 204/403.1

(57) ABSTRACT

Membranes useful for amperometric sensors are described. The membranes allow continuous and real time in vivo measurements of a variety of redox active chemical species present in a fluid sample. In some embodiments, the membrane comprises a redox mediator, a redox reactive species, and conductive nano structures, such as carbon nanotubes. The membrane can be provided on a working electrode of the sensor. Amperometric sensors incorporating the membranes and methods of treatment using the sensors are also described.

### CROSS-REFERENCE TO RELATED APPLICATIONS

This application claims the benefit of copending U.S. Provisional Application Ser. No. 61/050,550, filed May 5, 2008 and incorporates this provisional application by reference in its entirety.

### FIELD

Membranes that can be used with electrodes for reduction/oxidation-based sensors are disclosed. More particularly, membranes useful in amperometric sensors are disclosed.

### BACKGROUND

Materials and devices useful for analysis of chemical species in liquids are known in the art and can generally comprise wet chemistry or dry chemistry systems. Wet chemistry analyses employ reagents in liquid solution and are widely used in both manual and automated analytical methods. In dry chemistry systems, a complete chemistry for a particular analysis is provided on a single test probe or device and typically, no prior reconstitution of reagents is required. Dry chemistry methods are often simpler in design, require less reagent manipulation, provide quicker results, and exhibit superior stability.

Dry chemistry tests based on enzyme-catalyzed reactions have particularly grown in favor, one example being test strips used for the measurement of blood glucose levels. Such devices generally consist of a flat strip including a reaction layer supported on an inert film carrier and consisting of glucose oxidase enzymes coupled to a reduction/oxidation (redox) mediator dye. Blood glucose is analyzed by placing a sample of whole blood on the surface of the strip, allowing time for the underlying reactions to proceed, removing excess sample, and measuring the color development by spectroscopy methods. Such systems are effective but suffer from the limitation of single use measurements, after which disposal of the device is required.

An alternative to such test strips is electrochemical dry chemistry enzyme test devices. By electrically wiring enzymes as electrochemical biosensors and bioelectronic devices, it is possible to get both the recognition properties typical of the biological systems and the high sensitivity of electrochemical transducers to provide highly effective sensors. Such amperometric test devices typically include a sensing element comprising a measuring (working) electrode and a reference electrode. The electrodes are coated with appropriate reagents (such as an enzyme and an optional mediator) and are generally further coated with a cover membrane to prevent interfering species from reacting at the measuring electrode. When an appropriate test potential is applied, the measuring electrode provides a faradaic current proportional to the concentration of the chemical species being determined.

To allow for continuous measurement, the reagents must be bound into a membrane. Absent such binding, the reagents will simply be washed away from the electrode by a flowing sample. When mediators are bound in a membrane, however, their proximity to the other reactants is fixed, and the mediators cannot diffuse toward and away from the electrode to fully perform their function. Thus, the ability to cycle between reduced and oxidized forms is lost, and a continuous measurement is hindered or prevented.

Mediators play a crucial role in biological sensors, particularly amperometric sensors. Since the direct electrical communication between the active center of an enzyme (or other redox reactive species) and the surface of the test electrode is often kinetically prohibited, artificial redox mediators are usually employed to shuttle electrons between the enzyme and the electrode. This particularly overcomes any dependence on oxygen as a natural mediator, which can be a variable (as in the difference in oxygen tension of venous and arterial blood). Mediator reagent systems typically work best when the reaction components are free in solution. As noted above, though, continuous measurement devices require binding of the reagents in a membrane.

One attempt in the art to overcome this dichotomy is to simply bind the redox mediator and the test enzyme directly to the surface of an electrode. While this precludes the need for diffusion through a membrane to the electrode, this also limits the amount of current that can be measured.

Another method that has been attempted in the art is to attach the redox mediator to a conductive polymer. This approach provides only limited success because it requires exact synthesis of a macromolecule, which is often difficult to achieve and requires specialized conditions. The physical properties of the resulting polymer become secondary to the requirements of the electrochemistry and cannot be adjusted easily. Accordingly, it becomes difficult to make adjustments as required to accommodate needs, such as flexibility, sterilization, adhesion, and the like.

Various further approaches have also been used to immobilize the reagents used in amperometric sensors, such as adsorption, covalent bonding, physical entrapment in sol-gel materials, carbon-paste electrodes, and conducting or redox organic polymers. These methods, however, also suffer from various drawbacks.

### SUMMARY

A membrane for use in amperometric sensors is described herein that allows for continuous measurement of a redox active chemical species in a liquid mixture by providing the reactants used to detect the redox active chemical species. The membranes also provide for continuous measurement while still providing excellent sensitivity and signal strength. The membranes can be combined with a variety of electrodes to provide amperometric sensors. The application further describes methods of preparing such sensors and methods of treatment using such sensors.

In some embodiments, the membrane can be used with sensor electrodes. The membrane provides a low cost, efficient avenue for combining the necessary reactants of an electrochemical dry chemistry test for detecting a redox active chemical species, the reactants being provided in a manner that prevents substantial solubilization or washing away of the reactants in a test sample, particularly a flowing test sample. Further, the membrane comprises components useful to facilitate proper electron transmission from the redox active chemical species to the electrode. The facilitating components can provide desirable surface properties that translate into highly useful bulk properties for increasing sensor performance.

In some embodiments, the membrane comprises a plurality of redox reactive species particles and a plurality of conductive carbon nanostructures such as nanotubes, that can be applied to a conductive surface to form a membrane for an electrode used for the measurement of an analyte in a fluid. The redox reactive species can be an enzyme such as a FAD-containing or an oxidase enzyme. The redox reactive species particles and the conductive nanostructures can be chemisorbed or physisorbed to one another. In some embodiments, the redox reactive species particles and the conductive nanostructures form a random structure on the surface of the conductive material. In some embodiments, the redox reactive species particles and the conductive nanostructures form a non-layered structure. A redox mediator can be used with the redox reactive species particles and the conductive nanostructures and can interact with the redox reactive species particles and the conductive nanostructures. In some embodiments, the membrane can include a ferrocene compound as the redox mediator, an enzyme as the redox reactive species, and nanotubes as the nanostructures.

A method of forming a material for use in a membrane for a sensor electrode can comprise dissolving a redox mediator in a solvent to form a solution; adding to the solution a plurality of conductive nanostructures; and removing the solvent to produce a solid material comprising the redox mediator and the conductive nanostructures. The method can further include the additional steps of suspending the solid material in a buffer solution to form a suspension; and adding to the suspension a redox reactive species. In some embodiments, the redox mediator can be chemisorbed to the carbon nanostructures. In some embodiments, the conductive nanostructures are chemically derivatized to react with the redox mediator to produce redox mediator modified conductive nanostructures.

A method of forming a membrane for an electrode can comprise combining a redox mediator and conductive nanostructures, suspending the redox mediator and conductive nanostructures to form a suspension, mixing a redox reactive species with the suspension, and applying the suspension to a surface of the electrode such as by casting the suspension on the surface of the electrode. The redox mediator and conductive nanostructures can be combined by dissolving a redox mediator in a solvent to form a solution, adding the conductive nanostructures to the solution; and evaporating the solvent to produce a solid material comprising the redox mediator and the conductive nanostructures. In some embodiments, the redox mediator can be covalently bonded to the conductive carbon nanostructures.

An intermediate for use in the preparation of a membrane for an electrode can comprise a redox mediator and a plurality of conductive nanostructures such as carbon nanotubes. The redox mediator can include a ferrocene compound. In some embodiments, at least a portion of the redox mediator is covalently bonded to the conductive nanostructures to produce redox mediator modified conductive nanostructures. In some embodiments, the redox mediator is chemisorbed to the conductive nanostructures.

A method of forming a sensor can comprise providing an electrode having a surface and comprising an electrically conductive material, providing a composition comprising a plurality of redox reactive species particles, a plurality of conductive nanostructures, and optionally a redox mediator; and coating at least a portion of the surface of the electrode with the composition to form a membrane. The method can further include cross-linking the coating on the membrane with a reagent such as glutaraldehyde.

A method for in vivo measurement of blood glucose levels in a subject can comprise providing a sensor comprising an electrode having a surface and comprising an electrically conductive material and a membrane at least partially coating the surface of the electrode. The membrane comprises a redox mediator, glucose oxidase, and conductive nanostructures such as carbon nanotubes. The method can include contacting the electrode with blood from the subject and measuring an electric current from the electrode generated by electron transfer from the glucose oxidase to the electrode to determine the blood glucose level of the subject. The sensor can be provided in association with a catheter. The electrode can be provided on a substrate and can include a first surface that is at least partially coated with the membrane and an opposing surface that is adjacent the substrate, which is in turn adjacent the catheter. In some embodiments, the catheter comprises a lumen and the sensor is provided in the catheter and communicates with the lumen. The blood glucose measurements can be real-time measurements and can be taken continuously.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 shows an amperometric biosensor in the form of a flex circuit having a working electrode coated with a membrane.

FIG. 2 is a magnified side cross-sectional view of the working electrode portion of the biosensor of FIG. 1.

FIG. 3 is a block diagram illustrating a potentiostat set-up useful for interfacing a sensor with a control and display system.

FIG. 4 is a plot of current versus time for glucose sensing using a sensor comprising an electrode in comparison with other electrodes.

FIG. 5 is a plot of current versus glucose concentration in a test sample using a sensor comprising an electrode in comparison with other electrodes.

### DETAILED DESCRIPTION

As used in the specification, and in the appended claims, the singular forms “a”, “an”, and “the” include plural referents unless the context clearly dictates otherwise. The term “comprising” and variations thereof as used herein is used synonymously with the term “including” and variations thereof and are open, non-limiting terms.

A membrane useful for electrodes such as those used in amperometric sensors can allow for continuous, efficient in vivo measurements of a variety of redox active chemical species. In particular, the membrane can be used in the amperometric sensing of various redox active chemical species present in a liquid biological sample.

In some embodiments, the membrane can be such as those used with sensor electrodes. The membrane can provide a low cost, efficient avenue for combining the reactants of an electrochemical dry chemistry test for detecting a redox active chemical species, the reactants being provided in a manner that prevents substantial solubilization or washing away of the reactants in a test sample, particularly a flowing test sample. Further, the membrane can comprise components useful to facilitate proper electron transmission from the redox active chemical species to the electrode. Particularly, the facilitating components can provide bulk properties that increase sensor performance.

The membrane comprises a plurality of redox reactive species particles and a plurality of conductive carbon nanostructures such as conductive carbon nanotubes. Furthermore, the membrane can also include a redox mediator. These three components have been found to work synergistically to provide improved performance in amperometric sensing devices by solving known limitations in the field related to mediator diffusion and mediator proximity to the active moieties of redox reactive species, such as enzymes.

The redox reactive species can include any compound capable of participating in a biological mechanism or otherwise reacting with another biological compound in a manner capable of causing electron transfer. The redox reactive species comprises a species reactive in a redox reaction (i.e., that is capable of being reduced and/or oxidized).

In some embodiments, the redox reactive species comprises a biomolecule. The term “biomolecule”, as used herein, refers to any chemical compound naturally occurring in a living organism. For example, the biomolecule can be an enzyme. Compounds possessing enzymatic activity can be used as many interactions including enzymes and their substrates result in a transfer of one or more electrons. One particular example is the glucose oxidase enzyme, which binds to glucose to aid in the breakdown thereof in the presence of water and oxygen into gluconate and hydrogen peroxide. Accordingly, in certain embodiments, the redox reactive species can include glucose oxidase or a glucose dehydrogenase, such as bacterial glucose dehydrogenase, which is a quinoprotein with a polycyclicquinone prosthetic group. Bacterial glucose oxidase can be obtained from various microorganisms such as Aspergillus species, e.g., Aspergillus niger (EC 1.1.3.4), type II or type VII. Bacterial glucose dehydrogenases can be obtained from various microorganisms, such as Acinetobacter calcoaceticus, Gluconobacter species (e.g., G. oxidans), and Pseudomonas species (e.g., P. fluorescens and P. aeruginosa). Alternatively, the redox reactive species can be a lactate oxidase or lactate hydrogenase.

Many oxidases exhibit redox reactivity arising from the presence of a co-factor, such as flavin adenine dinucleotide (FAD). Thus, in certain embodiments, the redox reactive species comprises an FAD-containing oxidase enzyme. The flavin group of FAD is capable of undergoing redox reactions accepting either one electron in each step of a two-step process or accepting two electrons at once. In the reduced forms (e.g., FADH and FADH₂), the flavin adenine dinucleotide compound is capable of transferring electrons to other compounds or conductive materials. Non-limiting examples of FAD-containing enzymes that can be used include glucose oxidase, lactate oxidase, monoamine oxidase, D-amino acid oxidase, xanthine oxidase, and Acyl-CoA dehydrogenase.

In some embodiments, the enzyme is an oxidase enzyme and/or a flavin adenine dinucleotide (FAD) containing enzyme. For example, the enzyme can include a FAD-containing glucose oxidase enzyme. The enzyme can be provided in particulate form such as a lyophilized powder.

The membranes can be used with amperometric biosensors and can allow for detection and measurement of virtually any redox active chemical species present within a sample. This specifically extends to in vivo measurements of various compounds present in living subjects. Accordingly, the redox reactive species present in the membrane can be any compound capable of coupling with another compound (such as another species) in a redox reaction. For clarity, the example of glucose oxidase reacting with glucose is described herein although other analytes can be measured. Thus, the membrane can be customized for use in electrochemically detecting and measuring any analytes produced or otherwise present within a living subject by selecting the appropriate redox reactive species that will interact with the analyte of interest in a redox reaction. Clearly, this includes enzyme/substrate interactions, but also encompasses many further biochemical interactions.

In some embodiments, the redox reactive species component of the membrane is present in an amount sufficient to react with the desired redox active chemical species to be detected. In certain embodiments, the redox reactive species comprises about 30% by weight to about 80% by weight of the membrane, based on the overall weight of the membrane. In some embodiments, the redox reactive species comprises about 40% by weight to about 75% by weight, about 45% by weight to about 70% by weight, or about 50% by weight to about 70% by weight, based on the overall weight of the membrane.

The conductive carbon nanostructures can be conductive carbon nanotubes, nanospheres, nanosheets, other nanostructures, or mixtures thereof. In some embodiments, the nanostructures can be nanotubes. The nanostructures can be hydrophilic although nanostructures that are generally hydrophobic can also be used. Moreover, other conductive materials could also be used, such as metal colloids, particularly those that provide improved dimensionality.

The conductive nanostructures facilitate the transportation of electrons to the surface of the conductive material of the electrode and can provide surface properties to the membrane that translate into useful bulk properties. In other words, the conductive nanostructures can facilitate bulk chemistry to the electrode surface. The carbon nanostructures are capable of improving the surface properties of a membrane by providing an increased three-dimensional structure and thus work as dimensioning agents. The nanostructures can have inherent dimensions measurable in three planes (e.g., x, y, and z planes) wherein at least one of the x, y, or z dimensions is significantly different from and/or greater than the dimensions of the remaining membrane components. In some embodiments, the conductive carbon nanostructures are nanotubes. A carbon nanotube is a one-atom thick sheet of graphite rolled up into a seamless cylinder. Nanotubes can be in the form of single-walled nanotubes or multi-walled nanotubes. Single-walled nanotubes (SWNT's) can have a diameter of close to 1 nanometer, with a tube length that can be many thousands of times longer, and single-walled nanotubes with lengths up to orders of centimeters have been produced. Multi-walled nanotubes (MWNT's) include multiple layers of graphite rolled in on themselves to form a tube shape. There are two models that can be used to describe the structures of MWNT's. In the first model, sheets of graphite are arranged in concentric cylinders. In the second model, a single sheet of graphite is rolled in around itself, resembling a scroll of parchment or a rolled up newspaper. The interlayer distance is close to the distance between graphene layers in graphite. The carbon nanotubes can include single-walled nanotubes, multi-walled nanotubes, or mixtures thereof.

As carbon nanotubes are generally tubular in shape, they can be described in terms of their length and their diameter. The carbon nanotubes can have a diameter in the order of 1-2 nanometers (the average diameter can be from about 1.2-1.4 nm). The length-to-diameter ratio can be greater than 1000:1 and can exceed 10,000:1. In some embodiments, when combined with the remaining membrane components, the nanotubes will take on a random and/or non-layered placement within the membrane matrix.

The carbon nanostructures can improve the dimensionality of the membrane surface and lead to improved current generated at the interface. In the absence of nanostructures such as nanotubes, active redox turnover only happens at the original surface of the electrode. However, by includes the carbon nanostructures, active chemistry can occur all along the nanostructures. For examples, in embodiments that use nanotubes, active chemistry can occur along the edges of the nanotubes. Thus, the carbon nanostructures can increase the current generated by the electrode and thereby improve the readability and sensitivity of the sensor device.

In embodiments where carbon nanotubes are used in the membrane, a portion of the remaining membrane components can become trapped within the interior space of the nanotubes. In practical use, such as in aqueous environments (e.g., blood), the redox mediator can be maintained within the nanotube because of the extreme hydrophobic nature of certain mediators, such as ferrocenes. Thus, the reactants can move within the nanotube, which is in turn provided within the membrane but has direct contact with the surrounding environment. Moreover, the conductive nature of the nanotubes can further facilitate the transfer of electrons between the reactants.

In some embodiments, the conductive carbon nanostructures of the membrane are present in an amount sufficient to improve the bulk characteristics of the membrane. In certain embodiments, the conductive carbon nanostructures comprise about 1% by weight to about 50% by weight of the membrane, based on the overall weight of the membrane. In some embodiments, the redox mediator comprises about 1% by weight to about 40% by weight, about 2% by weight to about 35% by weight, or about 5% by weight to about 30% by weight, based on the overall weight of the membrane.

In some embodiments, the nanostructures can be chemically derivatized to increase the direct interaction between the nanostructures and the redox mediator such as by making the nanostructures reactive with the redox mediator. Chemical derivatization can also facilitate the suspension of the nanostructures in the aqueous system. The nanostructures can be chemically derivatized, for example, through the addition of a carboxylic moiety. Suitable carboxylic acid modified SWNT's are available, e.g., from Sigma-Aldrich (Aldrich product number 652490). In some embodiments, an amino functional redox mediator could be used in the membrane and can be directly attached to a carboxylic acid modified carbon nanostructure by a carbodiimide reaction scheme.

In some embodiments, the redox reactive species particles and the conductive nanostructures are chemisorbed or physisorbed to one another. Chemisorption or chemical adsorption is an IUPAC recognized term and is adsorption in which the forces involved are valence forces of the same kind as those operating in the formation of chemical compounds and can include charge transfer between compounds. Physisorption or physical adsorption is also an IUPAC recognized term and is adsorption in which the forces involved are intermolecular forces (such as van der Waals forces) and which do not involve a significant change in the electronic orbital patterns of the species involved.

The conductive nanostructures are provided in a sufficient amount and the conductive nanostructures are in sufficient contact with one another in the sensor to allow the conductive nanostructures to transfer electrons throughout the sensor and particularly from the redox reactive species to the underlying electrically conductive surface. The conductive nanostructures can transfer electrons from the redox reactive species to the underlying electrically conductive surface without the need to envelop the conductive nanostructures in a polymer such as a conductive or a redox polymer. Thus, the conductive nanostructures are not immobilized in the sensor by using a polymer such as a conductive or redox polymer.

In some embodiments, the redox reactive species particles and the conductive nanostructures (e.g. nanotubes) can form a random and non-layered structure on the surface of the conductive material. This random, non-layered structure is the result of mixing the nanostructures and redox reactive species particles together using a solution and merely allowing the solution to dry on a surface, such as the surface of a conductive material to form an electrode. In other words, the redox reactive species particles and the conductive nanostructures are not formed into an organized, layered structure. As a result, the membrane is relatively easy to apply to an electrode surface.

In some embodiments, a redox mediator is used with the redox reactive species particles and the conductive nanostructures and interacts with the redox reactive species particles and the conductive nanostructures. The redox mediator of the membrane can be any small molecule (i.e. non-polymeric) material capable of functioning as an electron shuttle. Useful redox mediators include materials that are reversible in nature and thus capable of alternating between reduced and oxidized states. The redox mediator can also reduce the oxygen sensitivity of the electrode membrane.

In certain embodiments, the redox mediator comprises ferrocene or a ferrocene derivative. Ferrocene is a metallocene compound comprising an iron atom “sandwiched” between two cyclopentadienyl rings. It is an electroactive organometallic compound acting as a pH-independent reversible electron donor. Ferrocene is amenable to derivatization with various substituents on the ring structure, including derivatization to be in polymer form, and such derivatives can differ in multiple properties, including redox potential, aqueous solubility, and bonding constant to various enzymes. Non-limiting examples of ferrocene derivatives that can be used include dimethyl ferrocene, 1,1′-ferrocene dicarboxylic acid, polyvinyl ferrocene (e.g., average molecular weight of about 16,000 Da), acetyl ferrocene, propioloyl ferrocene, butyryl ferrocene, pentanoyl ferrocene, hexanoyl ferrocene, octanoyl ferrocene, benzoyl ferrocene, 1,1′diacetyl ferrocene, 1,1′-dibutyryl ferrocene, 1,1′-dihexanoyl ferrocene, ethyl ferrocene, propyl ferrocene, n-butyl ferrocene, pentyl ferrocene, hexyl ferrocene, 1,1′-diethyl ferrocene, 1,1′-dipropyl ferrocene, 1,1′-dibutyl ferrocene, 1,1′-dihexyl ferrocene, cyclopentenyl ferrocene, cyclohexenyl ferrocene, 3-ferrocenoyl propionic acid, 4-ferrocenoyl butyric acid, 4-ferrocenylbutyric acid, 5-ferrocenylvaleric acid, 3-ferrocenoyl propionic acid esters, 4-ferrocenoyl butyric acid esters, 4-ferrocenyl butyric acid esters, 5-ferrocenylvaleric acid esters, dimethylaminomethyl ferrocene, and mixtures thereof.

Other materials that are also useful as redox mediators can be used in place of or in combination with ferrocene or ferrocene derivatives. For example, the redox mediator can include: bipyridinium salts and derivatives such as viologens; quinones such as benzoquinones (e.g., chloranil, fluoranil, and bromanil) or quinone-based biomolecules (e.g., vitamin K); osmium complexes; and phenazine compounds (e.g., alkyl-substituted phenazine derivatives). The redox mediator can be generally water-insoluble so it does not wash away during use.

The redox mediator component of the membrane can be present in an amount sufficient to facilitate electron transfer, as described herein. In certain embodiments, the redox mediator component comprises about 0.5% by weight to about 20% by weight of the membrane, based on the overall weight of the membrane. In further embodiments, the redox mediator comprises about 1% by weight to about 15% by weight, about 2% by weight to about 12% by weight, or about 3% by weight to about 10% by weight, based on the overall weight of the membrane.

The redox mediator can be chemisorbed or physisorbed to one or more of the redox reactive species particles and the carbon nanostructures. For example, the redox mediator can be chemisorbed to the carbon nanostructures in the membrane. Alternatively, the redox mediator and the conductive carbon nanostructures can be more closely associated to improve mediation. For example, the redox mediator can be covalently bonded to the conductive carbon nanostructures by derivatizing the conductive carbon nanostructures as described above and covalently bonding the nanostructures and the redox mediator to produce a redox mediator modified conductive nanostructures. In light of the increased dimensionality provided by the carbon nanostructures, the covalently attached redox mediator typically has increased contact with the surrounding environment, which increases the effectiveness of a sensor incorporating the membrane. The redox mediator modified conductive nanostructures can be prepared and isolated as intermediates for use in the preparation of a membrane. The intermediates are solids and can be provided in defined amounts for admixture with further components useful in preparing a membrane for an electrode.

In some embodiments, it may be useful to add further components to the membrane, e.g., to further stabilize the membrane and reduce washing away of the membrane or any of the components thereof. In some embodiments, the membrane is at least partially cross-linked using a reagent such as glutaraldehyde. Glutaraldehyde is particularly useful for inducing cross-linking between proteins by reacting with free amino groups in the redox reactive species through the formation of Schiff bases. The addition of glutaraldehyde in the membrane can be useful for causing cross-linking particularly when the redox reactive species is a protein, such as an enzyme. As a result, the other components of the membrane can be trapped within the cross-linked matrix and stabilized therein but generally do not participate in the cross-linking. The resulting cross-linked matrix is stable toward dissolution in the presence of a fluid at a neutral pH such as blood.

Various further components can be included in the membrane. For example, the membrane can further include stabilizing agents. Such stabilizing agents are useful to further reduce washing away of reactants from the membrane.

In some embodiments where the membrane is used in electrodes for glucose sensors, the membrane can include oxidase enzyme particles that are chemisorbed or physisorbed to conductive carbon nanostructures, and a redox mediator interacting with the enzyme particles and the conductive carbon nanostructures. The redox mediator can be either chemisorbed to the conductive carbon nanostructures or covalently bonded to derivatized conductive carbon nanostructures. The oxidase enzyme particles can be at least partially cross-linked using a cross-linking agent such as glutaraldehyde.

The material used to form the membrane can be produced by first dissolving the redox mediator in a solvent to form a solution, adding to the solution a plurality of conductive carbon nanostructures, and removing the solvent such as through evaporation to produce a solid material comprising the redox mediator and the conductive carbon nanostructures. The resulting intermediate solid material including the redox mediator and a plurality of conductive nanostructures can be isolated and then further combined with other membrane components for preparation of the membrane. In the event the conductive carbon nanostructures are derivatized to be reactive with the redox mediator, the redox mediator modified conductive carbon nanostructures can be isolated as the solid material. By covalently bonding the redox mediator to the carbon nanostructures prior to combining these components with the redox reactive species, a single component can be provided in a stable, solid form to be provided for later use with the redox reactive species. Thus, multiple options are available to an end user that allow for preparation of various membranes and electrodes for sensing a variety of redox active chemical species.

The intermediate solid material can be suspended in a buffer solution to form a suspension and the redox reactive species added to the suspension. At least a portion of the redox mediator molecules can be covalently bonded to the conductive carbon nanostructures to form redox mediator modified conductive carbon nanostructures, e.g., by using derivatized carbon nanostructures such as nanotubes and allowing the redox mediator and carbon nanostructures to react when combined. The derivatized conductive carbon nanostructures can be covalently bonded to the redox mediator prior to combining the redox mediator modified conductive carbon nanostructures with the redox reactive species.

In some embodiments, a membrane for an electrode can be produced by combining a redox mediator and conductive carbon nanostructures, suspending the redox mediator and conductive nanostructures to form a suspension, and mixing a redox reactive species with the suspension. The redox mediator and conductive carbon nanostructures can be combined by dissolving the redox mediator in a solvent to form a solution, adding the conductive carbon nanostructures to the solution, and evaporating the solvent to produce a solid material comprising the redox mediator and the conductive carbon nanostructures. The redox mediator and the conductive carbon nanostructures can be combined using the procedure described above even if the conductive carbon nanostructures have not been derivatized to react with the redox mediator. In some embodiments, where the redox mediator is not used, the conductive carbon nanostructures can be suspended in a buffer solution without being premixed with the redox mediator and then the redox reactive species added to the suspension.

Once the redox mediator modified conductive carbon nanostructures or a mixture of the redox mediator and conductive carbon nanostructures are mixed with the redox reactive species in a liquid medium such as a buffer solution, the mixture can be applied to a surface, such as to the surface of an electrically conductive material to form a membrane for an electrode. For example, the suspension can be applied to the electrode surface by coating at least a portion of the surface with the composition and drying the composition to form the membrane.

Once the membrane material is applied to the electrode surface, the membrane material can be contacted with a cross-linking agent such as glutaraldehyde. The cross-linking agent can be used to cross-link the components in the membrane such as the redox reactive species to form a cross-linked matrix.

As mentioned above, the membrane provided on the electrode can have a random or non-layered structure. Furthermore, the redox reactive species particles, the conductive nanostructures, and the optional redox mediator are typically not bound to the surface of the electrically conductive material that forms the electrode. For example, these components are not bound to the conductive material through the use of covalent bonding, electrostatic interaction or spatial trapping. Nevertheless, the manner in which the components are combined prevents substantial solubilization or washing away of the reactants in a flowing test sample.

In some embodiments, it is advantageous not to bind the membrane components to the electrode surface as it can inhibit the interaction between the reactants in the membrane and the analytes of interest in the fluids being tested. However, in some embodiments, once the membrane is applied, a portion of the electrode and the membrane can be enveloped in a material to bind the membrane to the electrode within the sensor. For example, a polymeric material can be used as a coating to bind the membrane to the electrode.

The resulting electrode including the membrane material can be used in a sensor such as those used for the in vivo measurement of redox active chemical species levels in a fluid sample such as blood. As used herein, the term “redox active chemical species” refers to an analyte capable of being reduced or oxidized through interaction with a separate chemical moiety and thus participating in a redox reaction. The liquid biological sample or fluid sample can be a sample of biological material that is either naturally present in a liquid or fluid state (e.g., blood, saliva, and urine) or a sample of biological material that is capable of being solubilized or reconstituted to be in a liquid or fluid state. The sensor allows for real-time and continuous measurements of analyte levels and can be used not only to detect the presence of the analyte but also to determine the actual analyte levels in the fluid. The sensor can be responsive to at least one redox active chemical species present in the liquid mixture.

In some embodiments, the sensor can be provided in association with a catheter. The electrode can include a first surface that is at least partially coated with the membrane and an opposing surface that is adjacent the substrate, which is in turn adjacent the catheter. In some embodiments, the catheter comprises a lumen and the sensor is provided in the catheter and communicates with the lumen.

In some embodiments, the membrane can be used in a sensor for the real-time measurement of blood glucose levels in a subject. For example, the method can include providing a sensor comprising an electrode having a surface and comprising an electrically conductive material and a membrane at least partially coating the surface of the electrode. The membrane can comprise a redox mediator, a glucose specific enzyme such as a FAD-containing glucose oxidase, and conductive carbon nanostructures. The electrode can be contacted with blood from the subject and an electric current can be measured from the electrode generated by electron transfer from the enzyme to the electrode to determine the blood glucose level of the subject. The blood can be a flowing sample provided adjacent the electrode, e.g., through the use of catheter.

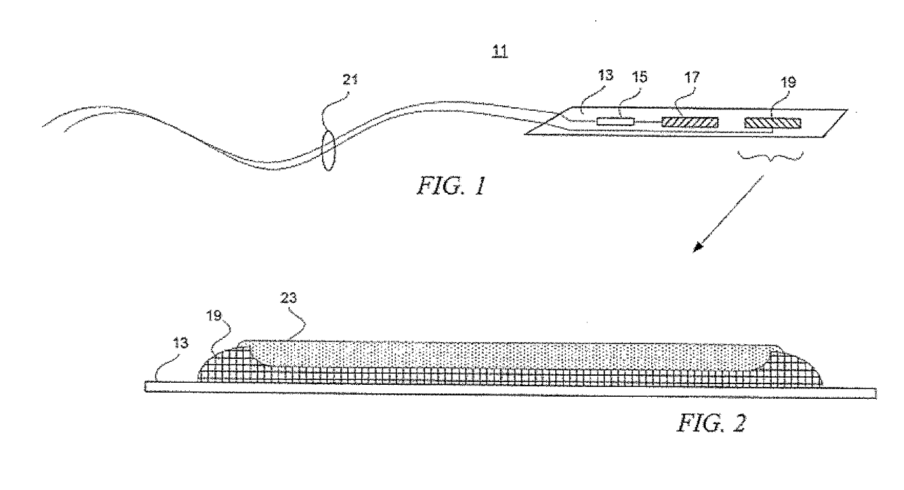

Turning to the drawings, FIG. 1 is a sensor 11 (such as a biosensor) in the form of a flex circuit that incorporates the membrane described herein. The biosensor 11 can be an amperometric sensor, such that a redox voltage is applied and a current is generated that is generally proportional to the amount of the redox active chemical species in the liquid test sample. The biosensor 11 can be formed on a substrate 13 (e.g., a flex substrate). One or more electrodes 15, 17 and 19 can be attached or bonded to a surface of the substrate 13. The biosensor 11 in FIG. 1 is shown with a reference electrode 15, a counter electrode 17, and a working electrode 19. In some embodiments, one or more additional working electrodes can be included on the substrate 13 and the biosensor 11 can include an enzyme sensor containing from 2 to 4 electrodes. The biosensor 11 at least includes a counter electrode 17 and a working electrode 19 and can also include the reference electrode 15. The reference electrode 15 is particularly useful for improving the accuracy of the sensor measurement. Furthermore, the addition of a second working electrode (not shown) can also further improve the accuracy of the sensor measurement.

The electrical wires 21 transmit power to the electrodes for sustaining an oxidation or reduction reaction, and can also carry signal currents to a detection circuit (not shown) indicative of a parameter being measured. The parameter being measured can be any redox active chemical species that occurs in, or can be derived from, blood chemistry. For example, the redox active chemical species can be hydrogen peroxide, formed from reaction of glucose with glucose oxidase, thus having a concentration that is proportional to blood glucose concentration.

The magnified cross-sectional side view of FIG. 2 shows a distal portion of the substrate 13 in the vicinity of the working electrode 19. The working electrode 19 can be at least partially coated with a membrane 23 including the conductive carbon nanostructures, the redox reactive species, and optionally the redox mediator. In some embodiments, the sensor is a glucose biosensor and the membrane 23 can include a FAD-containing oxidase enzyme as the redox reactive species.

The substrate 13 provides an insulated structure for mounting the electrodes and membrane layers and can be formed of a dielectric material such as a polyamide. In some embodiments, the substrate 13 can be between about 0.020 and 0.060 inches wide and between about 1.0 and 3.0 inches long. The thickness of the membrane layer can be between about 1 μm and 100 μm.

The electrical wires 21 can be coupled or soldered to conductive traces formed on the substrate 13 using flex circuit technology. For example, the traces can be gold-plated copper. The sensor 11 can be designed so that the flex circuit terminates to a tab that mates to a multi-pin connector, such as a 3-pin, 1 mm pitch ZIF Molex connector. Such a connection facilitates excitation of the working electrode and measurement of electrical current signals, for example, using a potentiostat or other controller.

The electrodes 15, 17 and 19 can be applied to the substrate 13 using a thick film process and commercially available inks. For example, the reference electrode 15 can be a silver/silver chloride type deposited or formed on the substrate 13. The reference electrode 15 establishes a fixed potential from which the potential of the counter electrode 17 and the working electrode 19 can be established.

The counter electrode 17 can be constructed from a conductive material such as platinum or graphite. These materials can be formulated as an ink for application to the substrate 13 using a thick film process and cured accordingly. The counter electrode 17 provides a working area for conducting the majority of electrons produced from the oxidation chemistry back to the solution. The working electrode 19 can be formed of an electrically conductive material such as platinum or graphite materials similar to those used for forming the counter electrode 17. Alternatively, the working electrode 19 can be formed from other conductive materials.

The sensor 11 can further include a thermal sensing element (not shown). As amperometric sensors are typically temperature sensitive, an uncompensated temperature change in the sensor can translate into measurement errors, and the thermal sensing allows the sensor to be compensated for temperature variations. Temperature variations are commonly due to the physiologic state of the test subject (or test liquid). For in vivo tests, the sensor can be placed near an infusion port so that the subsequent infusate passes by the sensor. By adding the temperature sensing element and measuring the sensor temperature, the temperature effect of the infusate can be minimized.

The sensors described herein can be useful for detecting and quantifying components of a liquid mixture. The sensor can be responsive to at least one redox active chemical species present in the liquid mixture. The sensors are particularly useful for in vivo measurements; however, they are not so limited and can also be used for various further methods. For example, the sensors can be used in various automated testing procedures where continuous, precise measurement of redox active chemical species in liquids is required, such as in a laboratory setting.

The reactive species used to carry out the electrochemical biosensing can be easily attached to the working electrode. Specifically, the membrane can be prepared by providing a composition for forming the membrane and coating at least a portion of the external surface of the electrode with the composition to form a membrane thereon. This method can also comprise further steps, such as contacting the coating of the membrane with a cross-linking agent.

Methods of treatment using the membranes and electrodes can provide for real-time measurement of a variety of analytes. For example, blood glucose levels in a subject can be measured in real time. The method can include the following steps: (a) providing a sensor comprising an electrode; (b) contacting the electrode with blood from the subject; and (c) measuring an electric current from the electrode.

In light of the variety of reactants available for use in the membrane, as described above, in vivo measurements can be made for a variety of compounds. For example, the real-time measurements of compounds reactive with oxidase enzymes such as FAD-containing oxidase enzymes can be made.

When the sensors and methods are used for in vivo testing in a live subject, placement of the sensor can be by any useful method known in the art using known devices, such as catheters. In these settings, the biosensor can function as an amperometric sensor while immersed in a patient's bloodstream. In some embodiments, catheters such as a multilumen catheter, a central venous catheter (CVC), a pulmonary artery catheter (PAC), a peripherally inserted central catheter (PICC), or other commonly used peripheral intravenous (IV) lines can provide a suitable platform for effective intravenous positioning of the biosensor. For example, the biosensor can be positioned in the patient's bloodstream by inserting a probe including the biosensor through a CVC or PAC or through a peripheral IV catheter or by using an introducer. One advantage of using a CVC or PAC for installing an intravenous biosensor is its ability to reach the largest blood vessels of the body where a biosensor can be exposed to an abundant flow of blood. Further, certain embodiments can be economically employed for use with multilumen catheters. Alternately, the sensor can be attached to a venous arterial blood management protection (VAMP) system by drawing a blood sample from the intravascular space and exposed to the sensor ex vivo.

In some embodiments, a sensor can be described as being in association with a catheter. In such embodiments, “association” comprises any method of combining a sensor and a catheter allowing for in vivo sensing using the sensor. For example, association can refer to direct attachment of the sensor to a surface of the catheter subject to ambient conditions. Association can also refer to a combination of the sensor and a catheter such that the sensor is directly adjacent the catheter (i.e., in a working proximity thereto) but not attached thereto. Still further, association can encompass placement of the sensor within a lumen of the catheter. Thus, association means that the sensor and the catheter are sufficiently related such that placement of the catheter in vivo likewise results in placement of the sensor in vivo.

In some embodiments, the electrode is provided on the substrate and comprises a first surface that is at least partially coated with the membrane and an opposing surface that is adjacent a first surface of the substrate. The opposing surface of the substrate can be provided adjacent the catheter. In some embodiments, the catheter comprises a lumen and the sensor is at least partially positioned within the lumen.

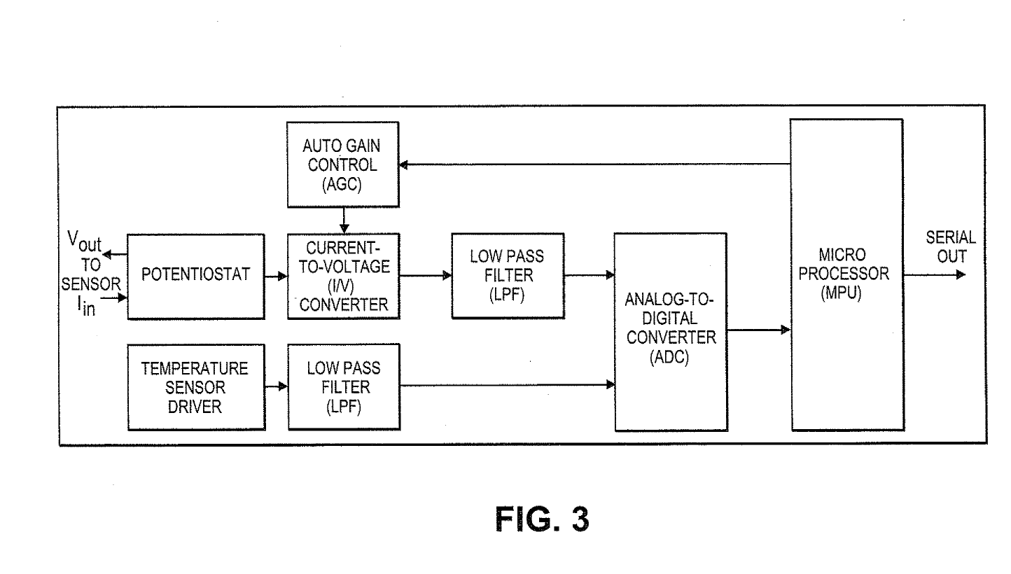

The sensors and methods can be used in connection with various types of instrumentation. For example, an instrument can include several components used to interface the sensor with a display. Such an interface is illustrated in the block diagram shown in FIG. 3. As illustrated therein, the potentiostat block applies a voltage to the sensor and measures the sensor response to the redox active chemical species in the sample being tested. The amperometric sensor can include an applied redox voltage to respond to the redox active chemical species. The potentiostat can apply a controlled voltage to the sensor and can measure the resultant current from the sensor. The measured current can be converted to a voltage signal in the current-to-voltage (I/V) converter. The instrumentation can include an auto gain control (AGC) component to control the amount of signal amplification necessary to convert the current to a suitable voltage signal.

After the signal has been converted to a voltage and amplified, the signal can pass through a low pass filter to remove unwanted noise. The voltage (analog) signal can be digitized in the analog-to-digital converter (ADC) and transmitted to the microprocessor (MPU). The MPU accumulates the different signals and converts them into an information packet, which is then sent out to a second controller. The MPU controls the AGC, which sets the amount of amplification in the I/V component. To improve the accuracy of the measurement, a temperature sensor can be applied to the sensor and can be a thermistor, thermocouple, or any temperature sensitive resistive element.

### Experimental

The following non-limiting examples are now provided.

### Example 1

### Preparation of Membrane Matrix

A mediator based membrane matrix was prepared by adding 3 mg of dimethyl ferrocene (DMFc) to 200 μL of tetrahydrofuran (THF). The DMFc was immediately solubilized. To this solution, 5 mg of carboxylic acid modified single-walled carbon nanotubes (SWNT-COOH) was added, and the mixture was vortexed and allowed to stand for approximately 1 hour. The resultant supernatant liquid was decanted, and the THF was evaporated using dry nitrogen. The remaining solids were transferred to a clean 2 mL vial and 1.0 mL of phosphate buffered saline (PBS) at pH 7.2 was added with 20 μL of TRITON® X100 nonionic surfactant. The mixture was sonicated for 20 minutes at room temperature to provide a stable suspension of DMFc modified SWNT-COOH in PBS. To this suspension, 25 mg of glucose oxidase per 500 μL of buffer was added. This was mixed gently until all enzyme solids appeared to dissolve (the mixture had an overall black appearance from the presence of the nanotubes) to yield the membrane matrix.

### Example 2

### Preparation of Membrane Coated Electrode

A trace amount of the membrane matrix from Example 1 was dispensed onto a graphite working electrode and allowed to air dry for 5 minutes to form a preliminary membrane. A trace amount of a 25% glutaraldehyde aqueous solution was then dispensed onto the membrane to cross-link the enzyme layer. The membrane was allowed to cure overnight to yield the final membrane coated electrode.

### Example 3

### Glucose Response

The membrane coated electrode from Example 2 was attached to a potentiostat for use in detecting glucose in a sample. The electrode was polarized at 170 mV using a graphite counter electrode with Ag/AgCl.

To test sensor response to glucose, 8 total membrane electrodes were prepared using various membrane matrix compositions. The membrane composition for each electrode is summarized below in Table 1.

TABLE 1

| Electrode | Chart | SWNT- | Glucose |
|---|---|---|---|
| No. | Reference | COOH | Oxidase |
| 1 | Ka60 | No | No |
| 2 | Kb24 | No | No |
| 3 | Kb25 | No | Yes |
| 4 | Kb26 | No | Yes |
| 5 | Kaxx | Yes | No |
| 6 | Kb28 | Yes | No |
| 7 | Kb29 | Yes | Yes |
| 8 | Kb30 | Yes | Yes |

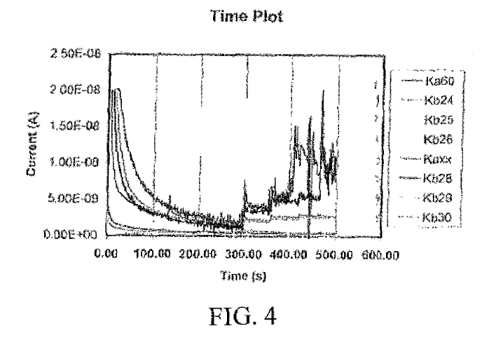

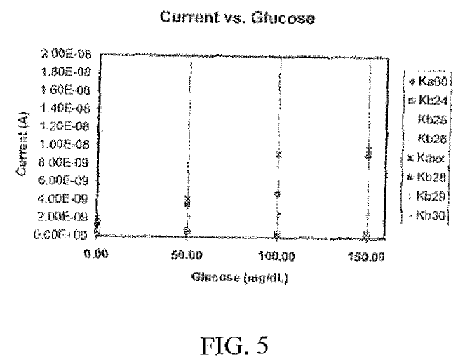

A plot of current versus time for all electrodes is provided in FIG. 4. A plot of current versus glucose concentration in the test sample is provided in FIG. 5.

As illustrated in FIG. 4 and FIG. 5, electrodes 1-4 (prepared without nanotubes) did not appear to respond to glucose while electrodes 5-8 (prepared with nanotubes) did exhibit a response. Accordingly, the addition of SWNT-COON measurably improved current output of the electrodes. Moreover, electrodes 7 and 8 showed a superior signal to noise ratio.

A number of embodiments have been described. Nevertheless, it will be understood that various modifications may be made without departing from the spirit and scope of the invention. Further, while only certain representative combinations of the formulations, methods, or products are specifically described, other combinations of the method steps or combinations of elements of a composition or product are intended to fall within the scope of the appended claims. Thus a combination of steps, elements, or components may be explicitly mentioned herein; however, all other combinations of steps, elements, and components are included, even though not explicitly stated.

That which is claimed:

1. An electrode for the continuous measurement of an analyte in a fluid, comprising:
a conductive material having a surface;
a plurality of redox reactive species particles; and
a plurality of conductive carbon nano structures;
wherein said redox reactive species particles and said carbon nanostructures are chemisorbed or physisorbed to one another and provided on the surface of said conductive material.

2. The electrode of claim 1, wherein the nanostructures include nanotubes.

3. The electrode of claim 1, further comprising a redox mediator interacting with said redox reactive species particles and conductive nanostructures.

4. The electrode of claim 3, wherein said redox mediator is chemisorbed or physisorbed to one or more of said redox reactive species particles and said carbon nanostructures.

5. The electrode of claim 4, wherein the redox mediator is covalently attached to the carbon nanostructures.

6. The electrode of claim 1, wherein said redox reactive species particles and said nanostructures are not bound to the surface of said conductive material.

7. The electrode of claim 1, wherein said redox reactive species particles are not bound to said conductive material through the use of covalent bonding, electrostatic interaction or spatial trapping.

8. The electrode of claim 1, wherein said carbon nanostructures facilitate the transportation of electrons to the surface of the conductive material of the electrode.

9. The electrode of claim 3, wherein the redox mediator comprises a ferrocene compound.

10. The electrode of claim 9, wherein the redox mediator comprises dimethyl ferrocene.

11. The electrode of claim 1, wherein the redox reactive species comprises an enzyme.

12. The electrode of claim 11, wherein the redox reactive species comprises an oxidase enzyme.

13. The electrode of claim 11, wherein the redox reactive species comprises an FAD-containing oxidase enzyme.

14. The electrode of claim 11, wherein the redox reactive species comprises glucose oxidase.

15. The electrode of claim 1, wherein the conductive nanostructures are chemically derivatized to include a carboxylic moiety.

16. The electrode of claim 1, wherein the conductive carbon nanostructures are hydrophilic.

17. The electrode of claim 1, wherein at least the redox reactive species are at least partially cross-linked using a cross-linking agent.

18. The electrode of claim 17, wherein the cross-linking agent comprises glutaraldehyde.

19. The electrode of claim 1, wherein the redox reactive species particles are oxidase enzyme particles, the electrode further comprises a redox mediator interacting with said redox reactive species particles and conductive nanostructures, and the oxidase enzyme particles are at least partially cross-linked using a cross-linking agent.

20. The electrode of claim 1, wherein said redox reactive species particles and said nanostructures provide a random structure on the surface of said conductive material.

21. The electrode of claim 1, wherein the nanostructures provide a non-layered structure on the surface of said conductive material.
